(19) United States
(12) Patent Application Publication
(10) Pub. No.: US 2025/0195075 A1
(43) Pub. Date: Jun. 19, 2025

(54) Blood Flow Regulation Devices and Methods

(71) Applicant: Bard Peripheral Vascular, Inc., Franklin Lakes, NJ (US)

(72) Inventors: Matt Casiraro, Tempe, AZ (US); Andrzej J. Chanduszko, Chandler, AZ (US); Ericka J. Prechtel, Salt Lake City, UT (US)

(21) Appl. No.: 18/844,379

(22) PCT Filed: Mar. 14, 2022
(86) PCT No.: PCT/US2022/020219

§ 371 Date: Sep. 5, 2024

(51) Int. Cl. A61B 17/12; A61B 17/00; A61B 90/00; A61M 25/06

(52) U.S. Cl. CPC A61B 17/12109; A61B 17/00234; A61B 17/12036; A61B 90/06; A61M 25/0662; A61B 2017/00022; A61B 2017/00238; A61B 2017/003; A61B 2017/00367; A61B 2017/00477; A61B 2017/00893; A61B 2017/1205; A61B 2090/064; A61M 2205/0272; A61M 2205/3327; A61M 2205/3331; A61M 2205/50

(57) ABSTRACT

Disclosed herein is a blood flow restriction device, that includes an elongate member configured for advancement along a blood vessel, having an actuator at a proximal end and a restriction mechanism at a distal end. The restriction mechanism is configured to transition between a various states of blood-flow restriction that includes deploying and/or retracting one or more restriction members between a collapsed state and a distally expanded state by the actuator. One or more restriction members may be cone shaped having conical wall which may include a number of apertures extending therethrough. The restriction mechanism may be transitionable either discreetly or variably between two or more blood-flow restriction states. The actuator may include a microprocessor controlled electro-mechanical actuator coupled with a sensor configured to measure a blood flow parameter, and logic of the controller may adjust a blood flow restriction amount based on a signal from the sensor.

### BACKGROUND

Pulmonary edema, a medical emergency, is an accumulation of fluid in the lungs. Pulmonary edema is often caused by congestive heart failure. When the heart is not able to pump efficiently, blood can back up into the veins that take blood through the lungs. As the pressure in these blood vessels increases, fluid is pushed into the air spaces (alveoli) in the lungs. This fluid reduces normal oxygen movement through the lungs. These two factors combine to cause shortness of breath. Failure of the left side of the heart (left ventricle) causes blood to accumulate in the veins of the lungs (pulmonary veins), producing a dangerous rise in blood pressure within these veins. Sustained high pressure in the pulmonary veins eventually forces some fluid from the blood into the interstitial space and eventually to the surrounding microscopic air sacs (alveoli), which transfer oxygen to the bloodstream. As the alveoli fill with fluid, they can no longer provide adequate amounts of oxygen to the body. Symptoms, especially severe breathing difficulty, develop over the course of a few hours and may be life-threatening. Although the outlook for pulmonary edema is favorable if the underlying disorder is treated in a timely fashion, the overall outcome for the patient depends upon the nature of the underlying disorder. Adults at high risk for heart failure are most commonly affected.

Typical treatment for patients experiencing pulmonary edema as a result of chronic heart failure is the administration of diuretic drugs, designed to reduce preload, which is described as the mechanical state of the heart at the end of diastole, i.e., the magnitude of the maximal (end-diastolic) ventricular volume or the end-diastolic pressure stretching the ventricles.

Some patients do not respond adequately to diuretic therapy. There is some evidence that by temporarily reducing blood flow to heart the symptoms of fluid overload can be improved, leading to increased diuretic efficiency and further improvement to patient's condition. Disclosed herein are blood flow restriction devices and methods that address the foregoing.

### SUMMARY

Disclosed herein is a catheter that, according to some embodiments, includes an elongate member configured for advancement along a blood vessel, where the elongate member extends between a proximal end and a distal end. the elongate member includes an actuator disposed at the proximal end, and a restriction member disposed at the distal end. The restriction member operatively coupled with the actuator via an elongate connection member extending along the elongate member. The restriction member is configured to transition between a collapsed state and a deployed state in response to an action by the actuator. The restriction member defines a cone in the deployed state, where the cone defines a conical wall extending radially outward and distally away from an apex portion of the cone. The cone further defines a conical wall that includes a number of apertures extending therethrough, and the conical wall is configured to extend radially outward to a wall of the blood vessel so that blood flow through the blood vessel is constrained to pass through the number of apertures, where the number of apertures are sized to define a restriction of the blood flow.

In some embodiments, at least a portion of the restriction member includes an anti-thrombotic coating.

In some embodiments, the restriction member is disposed within a sheath of the catheter in the collapsed state, and the restriction member extends distally away from the distal end of the elongate member in the deployed state.

In some embodiments, the catheter is configured for placement within the superior vena cava of a patient, and when deployed within the superior vena cava, the restriction member restricts the blood flow through the superior vena cava by at least 90 percent.

In some embodiments, the catheter is configured for placement within the inferior vena cava of the patient, and when deployed within the inferior vena cava, the restriction member restricts approximately 50 percent of the blood flow through the inferior vena cava.

In some embodiments, the actuator is manually manipulated by the clinician to perform the action.

Also disclosed herein is a blood flow regulation device, according to further embodiments, that includes: (i) an elongate member configured for advancement along a blood vessel, the elongate member extending between a proximal end and a distal end; (ii) a blood-flow regulation mechanism disposed at the distal end of the elongate member; and an actuator disposed at the proximal end of the elongate member, the actuator operatively coupled with the regulation mechanism. The regulation mechanism is configured to transition between a non-restriction state and a blood-flow restriction state in response to an action by the actuator. The regulation mechanism includes a first regulation member operatively coupled with the actuator via a first elongate connection member extending along the elongate member and a second regulation member operatively coupled with the actuator via a second elongate connection member extending along the elongate member. In the non-restriction state, each of the first and second regulation members are disposed in a collapsed state, and in the blood-flow restriction state, each of the first and second regulation members are disposed in an expanded state, where the first regulation member is positioned proximal the second regulation member.

In some embodiments, at least a portion of the device includes an anti-thrombotic coating.

In some embodiments, the device is configured for placement within a superior vena cava of a patient, and the blood-flow regulation mechanism is configured to restrict at least 90 percent of the blood flow through the superior vena cava. In other embodiments, the device is configured for placement within an inferior vena cava of the patient, and blood-flow regulation mechanism is configured to restrict approximately 50 percent of the blood flow through the inferior vena cava.

In some embodiments, in the collapsed state each of the first and second regulation members is disposed within a sheath of the elongate member, and in the expanded state each of the first and second regulation members is distally extended away from the distal end of the elongate member.

In some embodiments, the blood-flow regulation mechanism is transitionable between a first blood-flow restriction state where the blood-flow regulation mechanism is configured to restrict the blood flow a first restriction amount and a second blood-flow restriction state where the blood-flow regulation mechanism is configured to restrict the blood flow a second restriction amount, and where the second restriction amount is different from the first restriction amount.

In some embodiments, the first blood-flow restriction state is defined by a first relative position between the first and second regulation members in the expanded state and the second blood-flow restriction state is defined by a second relative position between the first and second regulation members in the expanded state, where the second relative position is different from the first relative position.

In some embodiments, each of the first and second regulation members defines a hollow cone having a proximal apex portion and a distal open end in the expanded state.

In some embodiments, the second regulation member includes a number of second apertures extending through a second conical wall of the second regulation member, and the second conical wall is configured to extend radially outward to a wall of the blood vessel so that the blood flow is constrained to pass through the number of second apertures.

In some embodiments, in the second relative position, the second regulation member is at least partially disposed within the first regulation member so that at least portion of the number of second apertures is occluded by a first conical wall of the first regulation member.

In some embodiments, the first regulation member includes a number of first apertures extending through the first conical wall, and the second regulation member is disposed within the first regulation member so that the second conical wall overlaps the first conical wall. Further, in the first relative position, the second regulation member is rotatably positioned relative to the first regulation member so that the number of first apertures overlaps the number of second apertures a first overlapping amount, and in the second relative position, the second regulation member is rotatably positioned relative to the first regulation member so that the number of first apertures overlaps the number of second apertures a second overlapping amount, where the second overlapping amount is different from the first overlapping amount.

In some embodiments, in the first blood-flow restriction state, the second regulation member is in the expanded state such that a second conical wall of the second regulation member is radially expanded to define an annular second passageway between the second regulation member and the wall of the blood vessel, and the annular second passageway is sized to define the first blood-flow restriction amount.

In some embodiments, in the second blood-flow restriction state, the first regulation member is in the expanded state such that a first conical wall of the first regulation member is radially expanded to define an annular first passageway between the first regulation member and the wall of the blood vessel, and the annular first passageway is sized to define the second blood-flow restriction amount. In some embodiments, the second blood-flow restriction amount is greater than the first blood-flow restriction amount

In some embodiments, the second regulation member defines a bullet shape in the distal deployed state, and the bullet shape includes a cone shaped proximal portion having a proximally oriented apex and a cone shaped distal portion having a distally oriented apex.

In some embodiments, the blood-flow regulation mechanism is further transitionable to a third blood-flow restriction state configured to restrict the blood flow a third restriction amount, and the third restriction amount is less than the first restriction amount and the second restriction amount.

In some embodiments, the regulation mechanism further includes a third regulation member operatively coupled with the actuator via a third elongate connection member extending along the elongate member, and the third regulation member is positioned distal the first and second regulation members. Further, in the non-restriction state, the third regulation member is disposed in a collapsed state within the sheath, and in the third blood-flow restriction state, the third restriction member is disposed in an expanded state such that a third conical wall of the first regulation member is radially expanded to define an annular third passageway between the third regulation member and the wall of the blood vessel, where the annular third passageway is sized to define the third blood-flow restriction amount.

In some embodiments, the actuator includes a controller including controller logic, and an electro-mechanical actuating mechanism coupled between the controller and the first and second elongate connection members, where the actuator is configured to variably transition the regulation mechanism between the first blood-flow restriction state and the second blood-flow restriction state.

In some embodiments, the device further includes a sensor positioned along the elongate member and coupled with the controller, where the sensor configured to provide a signal to the controller based on one or more of a static pressure, a dynamic pressure, or a velocity of the blood within the blood vessel, and where the logic is configured to variably transition the regulation mechanism in response to the signal from the sensor.

Further disclosed herein is a method for restricting blood flow through a blood vessel, including: (i) inserting a catheter into the blood vessel, the catheter comprising a blood-flow regulation mechanism disposed at a distal end of the catheter, the regulation mechanism configured to facilitate a restriction of blood flow through the blood vessel; (ii) deploying a first regulation member of the regulation mechanism away from a collapsed state toward a radially expanded state to define a first blood-flow restriction amount; and (iii) deploying a second regulation member of the regulation mechanism away from a collapsed state toward a radially expanded state to define a second blood-flow restriction amount less than the first blood-flow restriction amount.

In some embodiments of the method, the blood vessel is a superior vena cava of a patient, and the first blood-flow restriction amount is at least 90 percent of the blood flow through the superior vena cava. In other embodiments of the method, the blood vessel is an inferior vena cava of the patient, and the first blood-flow restriction amount is approximately 50 percent of the blood flow through the inferior vena cava.

In some embodiments, the method further includes deploying a third regulation member of the regulation mechanism away from a collapsed state toward a radially expanded state to define a third blood-flow restriction amount that is less than the second blood flow restriction amount.

In some embodiments of the method, an outside diameter of each of the first, second, and third regulation members defines a respective annular blood-flow passageway in combination a wall of the blood vessel, and each passageway is sized to define each of the first, second, and third blood-flow restriction amounts.

Further disclosed herein is another method for regulating blood flow through a blood vessel, including: (i) inserting a catheter into the blood vessel, the catheter comprising a blood-flow regulation mechanism disposed at a distal end of the catheter; deploying a first regulation member of the regulation mechanism away from a collapsed state toward a radially expanded state, the first regulation member defining a first conical wall extending between a proximal apex portion and an open distal end; and deploying a second regulation member of the regulation mechanism away from a collapsed state toward a radially expanded state, the second regulation member defining a second conical wall extending between a proximal apex portion and an open distal end. The first conical wall includes a number of apertures, and the first conical wall is configured to extend radially outward to a wall of the blood vessel so that blood flow through the blood vessel passes through the number of apertures. Further, the first regulation member is positionable within the second regulation member to adjust an occlusion amount of the number of apertures by the second conical wall so as to restrict the blood flow through the blood vessel.

In some embodiments, the method further includes adjusting the position of the first regulation member with respect to the second regulation member between a first position and a second position to adjust the restriction of the blood flow between a first restriction amount and a second restriction amount, where the second restriction amount is different from the first restriction amount.

In some embodiments of the method, the catheter includes a sensor configured to measure a blood-flow parameter that includes one or more of a static pressure, a dynamic pressure, or a velocity of the blood, and the method further includes adjusting the occlusion amount of the number of apertures based on a blood flow parameter measurement.

These and other features of the concepts provided herein will become more apparent to those of skill in the art in view of the accompanying drawings and following description, which describe particular embodiments of such concepts in greater detail.

### DRAWINGS

FIG. 1A illustrates a first embodiments of a blood-flow restriction device disposed within a vasculature of a patient, in accordance with some embodiments.

FIG. 1B illustrates a detailed view of a distal portion of the blood-flow restriction device of FIG. 1A with the restriction mechanism disposed in a collapsed state, in accordance with some embodiments.

FIG. 1C illustrates a detailed view of a distal portion of the blood-flow restriction device of FIG. 1A with the restriction mechanism disposed in a partially deployed state, in accordance with some embodiments.

FIG. 1D illustrates a detailed view of a distal portion of the device 100 in a distal deployed state, in accordance with some embodiments.

FIG. 2A illustrates side view of another embodiment of a blood-flow restriction device in a ready-to-use state, in accordance with some embodiments.

FIG. 2B illustrates a detailed view of a distal portion of the blood-flow restriction device of FIG. 2A with the restriction mechanism disposed in a partially deployed state, in accordance with some embodiments.

FIG. 2C illustrates a detailed view of a distal portion of the blood-flow restriction device of FIG. 2A with the restriction mechanism disposed in a fully deployed state, in accordance with some embodiments.

FIG. 3A illustrates a side view of another embodiment of a blood-flow restriction device showing the restriction mechanism disposed in a partially deployed state, in accordance with some embodiments.

FIG. 3B illustrates a detailed view of a distal portion of the blood-flow restriction device of FIG. 3A with the restriction mechanism disposed in a fully deployed state, in accordance with some embodiments.

FIG. 4A illustrates side view of another embodiment of a blood-flow restriction device in a ready-to-use state, in accordance with some embodiments.

FIG. 4B illustrates a detailed view of a distal portion of the blood-flow restriction device of FIG. 4A with the restriction mechanism disposed in a first partially deployed state, in accordance with some embodiments.

FIG. 4C illustrates a detailed view of the distal portion of FIG. 4B with the restriction mechanism disposed in a second partially deployed state, in accordance with some embodiments.

FIG. 4D illustrates a detailed view of the distal portion of FIG. 4C with the restriction mechanism disposed in a fully deployed state, in accordance with some embodiments.

FIG. 5A illustrates side view of another embodiment of a blood-flow restriction device in a ready-to-use state, in accordance with some embodiments.

FIG. 5B illustrates a detailed view of a distal portion of the blood-flow restriction device of FIG. 5A with the restriction mechanism disposed in a partially deployed state, in accordance with some embodiments.

FIG. 5C illustrates a detailed view of the distal portion of FIG. 5B with the restriction mechanism disposed in a fully deployed state, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a blood-flow restriction device includes a portion or section of the blood-flow restriction device intended to be near a clinician when the blood-flow restriction device is used on a patient. Likewise, a “proximal length” of, for example, the blood-flow restriction device includes a length of the blood-flow restriction device intended to be near the clinician when the blood-flow restriction device is used on the patient. A “proximal end” of, for example, the blood-flow restriction device includes an end of the blood-flow restriction device intended to be near the clinician when the blood-flow restriction device is used on the patient. The proximal portion, the proximal section, or the proximal length of the blood-flow restriction device can include the proximal end of the blood-flow restriction device; however, the proximal portion, the proximal section, or the proximal length of the blood-flow restriction device need not include the proximal end of the blood-flow restriction device. That is, unless context suggests otherwise, the proximal portion, the proximal section, or the proximal length of the blood-flow restriction device is not a terminal portion or terminal length of the blood-flow restriction device.

With respect to “distal,” a “distal portion” or a “distal section” of, for example, a blood-flow restriction device includes a portion or section of the blood-flow restriction device intended to be near or in a patient when the blood-flow restriction device is used on the patient. Likewise, a “distal length” of, for example, the blood-flow restriction device includes a length of the blood-flow restriction device intended to be near or in the patient when the blood-flow restriction device is used on the patient. A “distal end” of, for example, the blood-flow restriction device includes an end of the blood-flow restriction device intended to be near or in the patient when the blood-flow restriction device is used on the patient. The distal portion, the distal section, or the distal length of the blood-flow restriction device can include the distal end of the blood-flow restriction device; however, the distal portion, the distal section, or the distal length of the blood-flow restriction device need not include the distal end of the blood-flow restriction device. That is, unless context suggests otherwise, the distal portion, the distal section, or the distal length of the blood-flow restriction device is not a terminal portion or terminal length of the blood-flow restriction device.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Unless defined otherwise, all technical and scientific terms used herein have the same meaning as commonly understood by those of ordinary skill in the art.

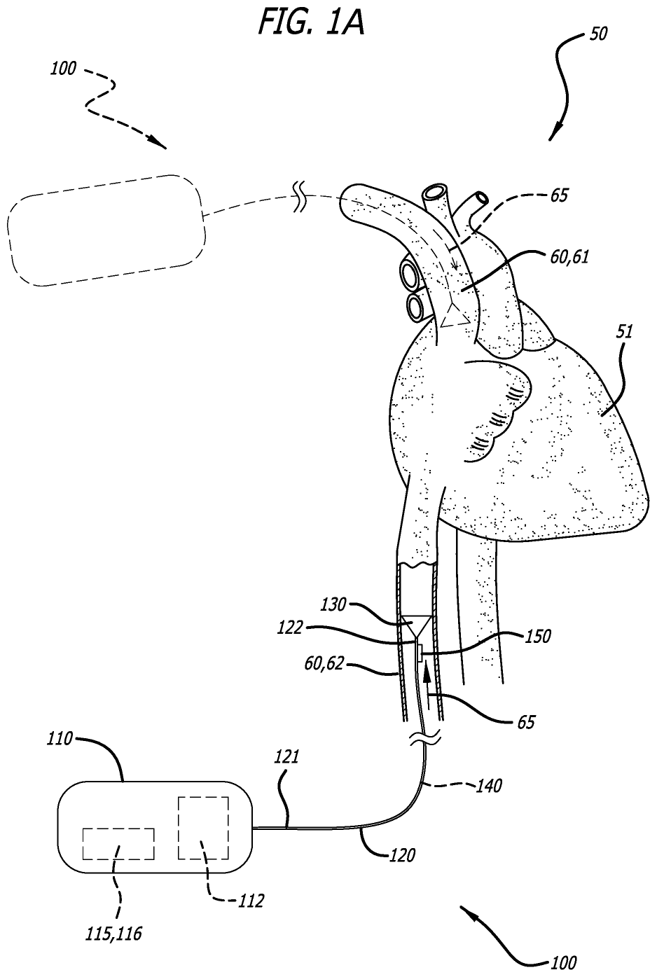

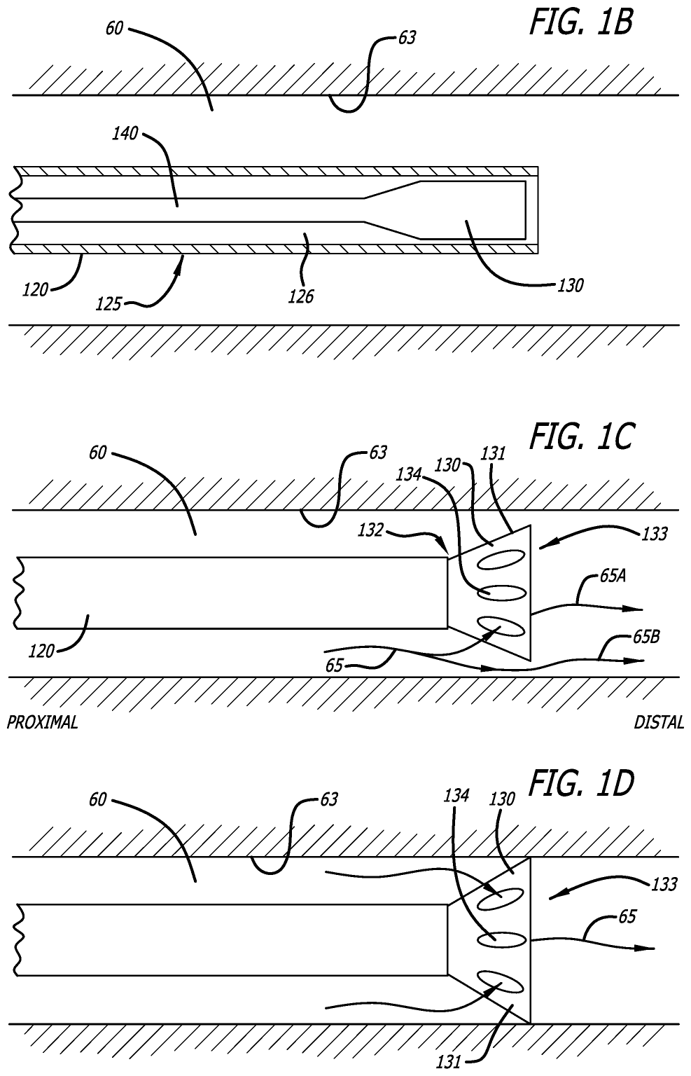

FIGS. 1A-1D illustrate a first embodiment of the blood-flow restriction device in use with a patient 50. The blood-flow restriction device (device) 100 is configured for insertion into the blood vessel 60. The device 100 is generally configured to selectively restrict blood flow 65 through the blood vessel 60 once the device 100 is inserted into the blood vessel 60. In FIG. 1A, the device 100 is shown inserted in the same direction as the blood flow 65. However, the device 100 may also be inserted into the blood vessel 60 in a direction opposite the flow. The device 100 may be a catheter, such as a central venous catheter, or a peripherally inserted central catheter, for example. In some instances of use, it may be desirable to reduce the total blood flow 65 to the heart 51 by about 30 percent. Typically, about 70 percent of the total blood flow 65 to the heart 51 flows through the inferior vena cava 62 and about 30 percent flows through the superior vena cava 61. The device 100 may be configured to restrict the total blood flow toward the heart 51 by about 30 percent.

FIG. 1A illustrates the device 100 disposed within the inferior vena cava 62 of the patient 50, in accordance with some embodiments. In some embodiments, the device 100 may be configured for insertion within the vasculature of the patient 50 so that a restriction mechanism 130 is disposed within the inferior vena cava 62. In such embodiments, the restriction mechanism 130 may be configured to reduce the blood flow 65 through the inferior vena cava 62 up to about 50 percent.

In other embodiments, as shown in phantom lines, the device 100 may be configured for insertion within the vasculature of the patient 50 so that the restriction mechanism 130 is disposed within the superior vena cava 61. In such embodiments, the restriction mechanism 130 may be configured to reduce the blood flow 65 through the superior vena cava up to and/or more than about 90 percent.

The device 100 generally includes an elongate member 120 configured for advancement along the blood vessel 60. The elongate member 120 extends between a proximal end 121 and a distal end 122. An actuator 110 is disposed at the proximal end 121 and is configured for placement outside of the patient 50. A restriction mechanism 130 disposed at the distal end 122 and is configured for placement within the blood vessel 60. The restriction mechanism 130 is operatively coupled with the actuator 110 via an elongate connection member 140 extending along the elongate member 120. The restriction mechanism 130 is configured to transition between a proximal collapsed state and a distal deployed state in response to an action by the actuator 110 as further described below. The elongate connection member 140 may be a wire, such as a guidewire, for example. The elongate connection member 140 have a solid or hollow cross section.

The actuator 110 may be a manually operated actuator 110. In other words, the actuator 110 may facilitate transitioning of the restriction mechanism 130 between the collapsed state and the deployed state manually by a clinician. As such, although not shown, the actuator 110 may include one or more levers, knobs, buttons, sliders or any other suitable devices configured to selectively deploy and retract the restriction mechanism 130 via the elongate connection member 140.

In some embodiments, the actuator 110 may include an electro-mechanical actuating mechanism 112 coupled with the elongate connection member 140. The electro-mechanical actuating mechanism 112 may be configured to longitudinally displace the elongate connection member 140 causing deployment and/or retraction of the restriction mechanism 130.

In some embodiments, the actuator 110 may further include a controller 115 coupled with the electro-mechanical actuating mechanism 112. The controller 115 may include a microprocessor, memory, and an interface comprising one or more digital/analog inputs and outputs. The controller maybe be configured to receive an input and provide an output control signal to the electro-mechanical actuating mechanism 112. In some embodiments, the controller may be a microcontroller, i.e., a small computer on a single integrated circuit comprising one or more CPUs (processor cores) along with memory and programmable input/output peripherals, where the memory includes logic 116 stored thereon. In some embodiments, the logic 116 may be configured to continually monitor an input signal and compare the input signal with one or more programmed limits as stored in memory.

In some embodiments, the device 100 may include sensor 150 configured to determine a parameter of the blood flow 65. The sensor 150 may located at any position along the elongate member 120 consistent with obtaining a measure of the parameter. The sensor is generally configured to provide a signal to the actuator 110, where the signal is related to the blood flow 65. The sensor may be configured to determine one or more of a static pressure, a dynamic pressure, or a velocity of the blood within the blood vessel 60. For example, the pressure up stream of the restriction mechanism 130 may increase in relation to a restriction amount. Similarly, the pressure downstream of the restriction mechanism 130 may decrease in relation to a restriction amount. By way of other examples, the sensor may measure a change in velocity or flow rate of the blood flow 65 directly.

In some embodiments, the logic 116 may be configured to monitor the signal from the sensor 150 and adjust the deployment of the restriction mechanism 130 in response to the signal from the sensor. For example, the actuator 110 may be configured to retract the restriction mechanism 130 either partially or totally in response to a pressure signal from the sensor 150 to inhibit distress or trauma of the patient.

FIG. 1B illustrates a detailed view of a distal portion of the device 100 within the blood vessel 60. As shown, the elongate member 120 includes a sheath 125 extending along the elongate member 120, where the sheath 125 defines a lumen 126. FIG. 1B illustrates the restriction mechanism 130 in a proximal collapsed state disposed with the lumen 126. The elongate connection member 140 is coupled with the restriction mechanism 130 so that longitudinal displacement of the elongate connection member 140, as may be defined by the actuator 110, is transferred to the restriction mechanism 130.

FIG. 1C illustrates a detailed view of the distal portion of FIG. 1B with the restriction mechanism 130 disposed in a partially deployed state, in accordance with some embodiments. As shown, the restriction mechanism 130 is distally displaced beyond the distal end 122 of the elongate member 120. The restriction mechanism 130 is expanded away from the collapsed state defining a cone shape where the restriction mechanism 130 defines a conical wall 131 extending radially outward toward the blood-vessel wall 63 and distally away from an apex portion 132 to distal open end 133 of the cone shape, and the apex portion 132 is coupled with the elongate connection member 140.

The conical wall 131 includes a number of apertures 134 (e.g., 1, 2, 3, 4 or more apertures) extending through the conical wall 131. In the partially deployed state, a first portion 65A of the blood flow 65 passes through the apertures 134 and a second portion 65B passes through an annular passageway between the restriction mechanism 130 and the blood-vessel wall 63.

FIG. 1D illustrates a detailed view of a distal portion of the device 100 in a distal deployed state, in accordance with some embodiment. As shown, the restriction mechanism 130 is radially expanded to fill the blood vessel 60, i.e., the conical wall 131 is radially extended to the blood vessel wall. Hence, a substantial entirety of blood flow 65 passes through the apertures 134. Constraining the blood flow 65 to pass through the apertures 134 may substantially define the blood-flow restriction.

The restriction mechanism 130 may be formed of any suitable materials and structure consistent with transitioning between the proximal collapsed state and the distal deployed state. In some embodiments, the restriction mechanism 130 may include a wire frame structure (not shown). The wire frame structure may be formed of any suitable wire material, such as stainless steel, or Nitinol, for example. The wire frame structure may consist of straight wire struts spaced evenly around a circumference or a helical shaped wire to produce a cone. In some embodiments, the restriction mechanism 130 may include a basket shape in lieu of a cone shape. The restriction mechanism 130 may further include a polymeric film defining the conical wall 131. The apertures 134 may be formed through the conical wall 131 via any suitable process, such as laser cutting, for example. In some embodiments, the structure of the restriction mechanism 130 may define a bias of the restriction mechanism 130 toward the distal deployed state (i.e., expanded state). In some embodiments, the wire frame structure and the polymeric film may be integrally formed of the same polymeric material.

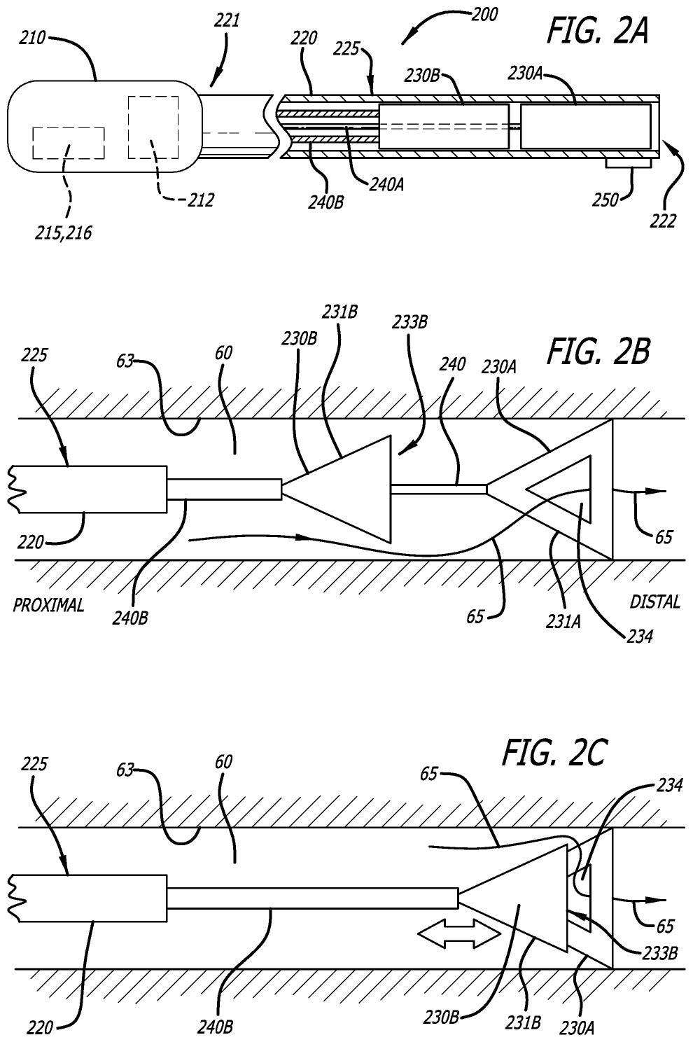

FIGS. 2A-2C illustrate another embodiment of a blood-flow restriction device that can, in certain respects, resemble components of the blood-flow restriction device described in connection with FIGS. 1A-1D. It will be appreciated that all the illustrated embodiments may have analogous features. Accordingly, like features are designated with like reference numerals with the leading digits incremented to “2.” For instance, the elongate member is designated as “120” in FIGS. 1A-1D, and an analogous elongate member is designated as “220” in FIGS. 2A-2C. Relevant disclosure set forth above regarding similarly identified features thus may not be repeated hereafter. Moreover, specific features of the blood-flow restriction device and related components shown in FIGS. 1A-1D may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the blood-flow restriction device of FIGS. 2A-2C. Any suitable combination of the features, and variations of the same, described with respect to the blood-flow restriction device and components illustrated in FIGS. 1A-1D can be employed with the blood-flow restriction device system and components of FIGS. 2A-2C, and vice versa. This pattern of disclosure applies equally to further embodiments depicted in subsequent figures and described hereafter.

FIG. 2A illustrates a side view of the blood flow regulation device (device) 200 in a ready-to-use state. The device 200 generally includes the actuator 210, a blood-flow restriction mechanism 230, and an elongate member 220 coupled between the actuator 210 and the blood-flow restriction mechanism 230. The blood-flow restriction mechanism 230 of the device 200 includes first restriction member 230A and a second restriction member 230B where each are shown in the proximal collapsed state within the sheath 225. The first restriction member 230A and the second restriction member 230B are operatively coupled with actuator 210 via a first elongate connection member 240A and a second elongate connection member 240B, respectively. Although not required, the first elongate connection member 240A may be disposed within a lumen of the second elongate connection member 240B.

FIG. 2B illustrates a distal portion of device 200 disposed within the blood vessel 60. The first restriction member 230A is disposed in the distal deployed state having a conical wall 231A radially expanded to the blood-vessel wall 63 so that the blood flow 65 passes through a number (e.g., 1, 2, 3, 4, or more) of apertures 234 extending through the conical wall 231A of the first restriction member 230A.

The second restriction member 230B is also disposed in partially deployed state (i.e., radially expanded state). In FIG. 2B the second restriction member 230B is displaced partially toward the first restriction member 230A.

FIG. 2C illustrates the distal portion of device 200 where the first restriction member 230A and the second restriction member 230B are each in the fully deployed state. As shown, a proximal portion of the first restriction member 230A is disposed through the distal open end 233B of the second restriction member 230B so that a portion of the first restriction member 230A is disposed within the second restriction member 230B. As such, the conical wall 231B of the second restriction member 230B overlaps the conical wall 231A of the first restriction member 230A so that the conical wall 231B may occlude a portion of the apertures 234. The conical wall 231B of the second restriction member 230B does not extend to the blood-vessel wall 63 leaving an annular passageway for the blood flow 65 to pass by the second restriction member 230B.

The device 200 may be configured to define two more blood-flow restriction states. For example, the device 200 may define a first blood-flow restriction state where the conical wall 231B does not occlude any portion of the apertures 234 or occludes a first portion of the 234. Similarly, the device 200 may also define a second blood-flow restriction state where the conical wall 231B entirely occludes the apertures 234 which may completely restrict the blood flow 65 or occludes a first portion of the 234. In some embodiments, the second portion may be greater than the first portion. Further, the device 200 may define one or more additional blood-flow restriction states between the first blood-flow restriction state and the second blood-flow restriction state.

The device 200 may also be configured to variably restrict the blood flow 65. For example, the actuator 210 may be configured to variably position the second restriction member 230B with respect to the first restriction member 230A so that the conical wall 231B variably occludes the apertures 234.

In some embodiments, the actuator 210 may include an electro-mechanical actuating mechanism 212 coupled with each of the elongate connection members 240A, 240B. The electro-mechanical actuating mechanism 212 may be configured to longitudinally displace the elongate connection members 240A, 240B causing deployment and/or retraction of the restriction members 230A, 230B. In some embodiments, the electro-mechanical actuating mechanism 212 may be configured to longitudinally adjust the elongate connection member 240A with respect to the elongate connection member 240B or vice versa to adjust the blood-flow restriction amount defined by the blood-flow restriction mechanism 230.

In some embodiments, the actuator 210 may further include a controller 215 coupled with the electro-mechanical actuating mechanism 212. In some embodiments, logic 216 of the controller 215 may be configured to continually monitor an input signal and compare the input signal with one or more programmed limits as stored in memory. In some embodiments, the device 200 may also include a sensor 250 configured to determine a parameter of the blood flow 65, where the sensor 250 is coupled with the controller 215. The logic 216 of the controller 215 may be configured to monitor a signal from the sensor 250 and adjust a blood-flow restriction amount defined by the blood-flow restriction mechanism 230 based on the signal from the sensor 250.

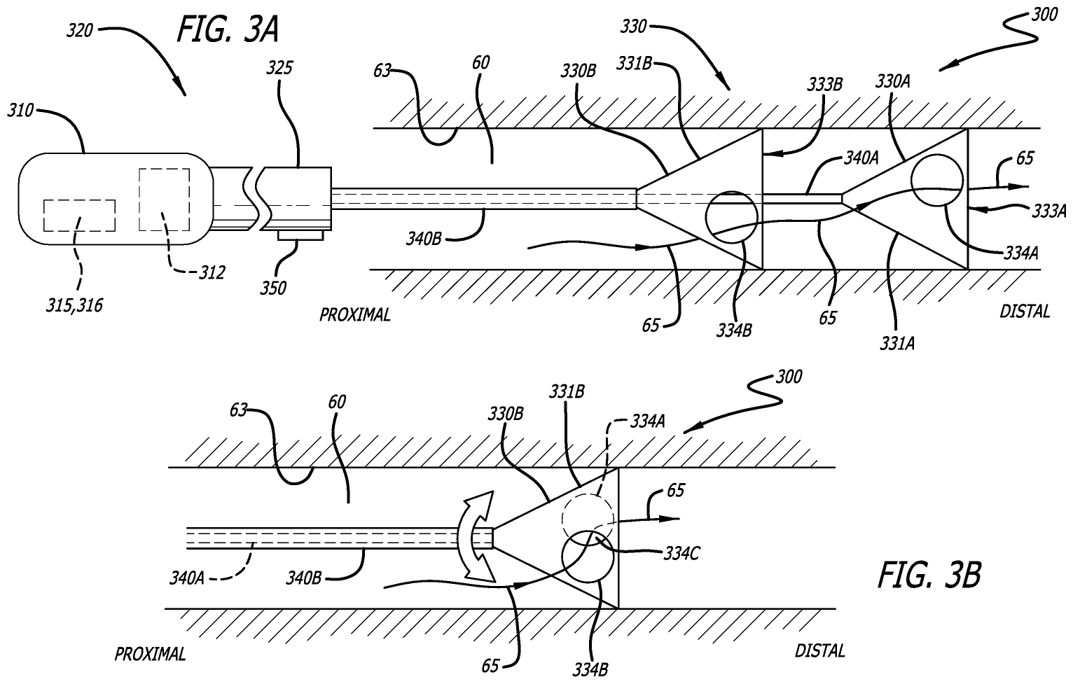

FIGS. 3A-3B illustrate another embodiment of a blood-flow restriction device. FIG. 3A illustrates a side view of the blood flow regulation device (device) 300. The device 300 generally includes the actuator 310 coupled with the elongate member 320. A blood-flow restriction mechanism 330 of the device 300 includes first restriction member 330A and a second restriction member 330B, where each are shown in the proximal collapsed state within the sheath 325. The first restriction member 330A and the second restriction member 330B are operatively coupled with the actuator 310 via a first elongate connection member 340A and a second elongate connection member 340B, respectively. Although not required, the first elongate connection member 340A may be disposed within a lumen of the second elongate connection member 340B.

The first restriction member 330A is disposed in the distal deployed state (i.e., radially expanded state). The conical wall 331A of the second restriction member 330A may also extend to the blood-vessel wall 63 so that a substantial entirety of the blood flow 65 passes through a number (e.g., 1, 2, 3, 4, or more) of apertures 334A extending through the conical wall 331A.

The second restriction member 330B is shown disposed in partially deployed state, i.e., positioned proximally away from the first restriction member 330A. When fully deployed, the conical wall 331B of the second restriction member 330B may also extend to the blood-vessel wall 63 so that a substantial entirety of the blood flow 65 passes through a number (e.g., 1, 2, 3, 4, or more) of apertures 334B extending through the conical wall 331B.

FIG. 3B illustrates the distal portion of device 300 where the first restriction member 330A and the second restriction member 330B are each in the fully deployed state. As shown, the first restriction member 330A is substantially disposed within second restriction member 330B. As such, the conical wall 331B overlaps the conical wall 331A. As shown, the first and second restriction members 330A, 330B are configured so that the apertures 334A, 334B may overlap each other to define an opening 334C extending through both conical walls 331A, 331B in accordance with a rotational position of the second restriction member 330B with respect to the first restriction member 330A.

The device 300 may be configured to define two or more blood-flow restriction states. For example, the device 300 may define a first blood-flow restriction state where the apertures 333A, 334B completely overlap each other defining a maximum flow area of the opening 334C. Similarly, the device 300 may also define a second blood-flow restriction state where apertures 333A, 334B are substantially misaligned defining a minimum flow area of the opening 334C which may substantially block the blood flow 65. Further, the device 300 may define one or more additional blood-flow restriction states between the first blood-flow restriction state and the second blood-flow restriction state.

The device 300 may also be configured to variably restrict the blood flow 65. For example, the actuator 310 may be configured to variably adjust the rotational position of the second restriction member 330B with respect to the first restriction member 330A so that the flow area of the opening 334C may be defined at any state between a fully open state and a fully closed state.

In some embodiments, the actuator 310 may include an electro-mechanical actuating mechanism 312 coupled with each of the elongate connection members 340A, 340B. The electro-mechanical actuating mechanism 312 may be configured to longitudinally displace the elongate connection members 340A, 340B causing deployment and/or retraction of the restriction members 330A, 330B. In some embodiments, the electro-mechanical actuating mechanism 312 may be configured to rotate the elongate connection member 340A with respect to the elongate connection member 340B or vice versa to adjust the blood-flow restriction amount of the blood-flow restriction mechanism 330.

In some embodiments, the actuator 310 may further include a controller 315 coupled with the electro-mechanical actuating mechanism 312. In some embodiments, logic 316 of the controller 315 may be configured to continually monitor an input signal and compare the input signal with one or more programmed limits as stored in memory. In some embodiments, the device 300 may also include sensor 350 configured to determine a parameter of the blood flow 65, where the sensor 350 is coupled with the controller 315. The logic 316 of the controller 315 may be configured to monitor a signal from the sensor 350 and adjust a blood-flow restriction amount of the blood-flow restriction mechanism 330 based on the signal from the sensor 350.

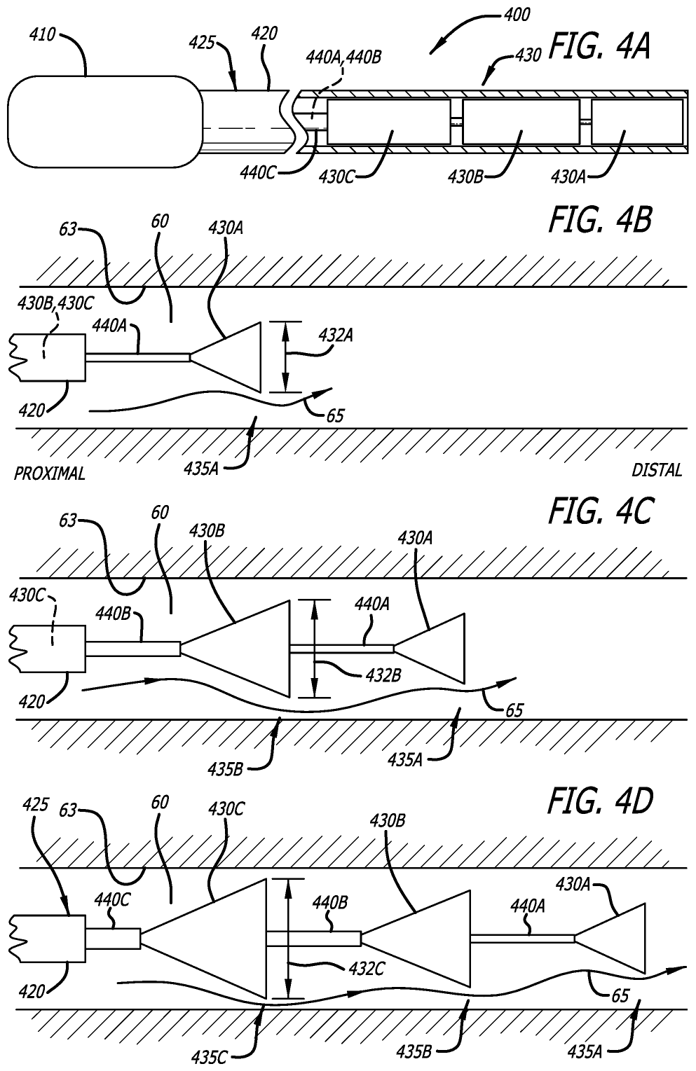

FIGS. 4A-4D illustrate another embodiment of a blood-flow restriction device. FIG. 4A illustrates a cross-sectional side view of the blood flow regulation device (device) 400 in a ready-to-use state. The device 400 generally includes the actuator 410, blood-flow restriction mechanism 430, elongate member 420 coupled between the actuator 410 and the blood-flow restriction mechanism 430. The blood-flow restriction mechanism 430 of the device 400 includes a first restriction member 430A, a second restriction member 430B, and a third restriction member 430C, where each is shown in the proximal collapsed state within the sheath 425. Each of the first restriction member 430A, the second restriction member 430B, and third restriction member 430C is operatively coupled with actuator 410 via a first elongate connection member 440A, a second elongate connection member 440B, and a third elongate connection member 440C, respectively. Although not required, the first elongate connection member 440A may be disposed within a lumen of the second elongate connection member 440B. Similarly, the second elongate connection member 440B may be disposed within a lumen of the third elongate connection member 440C.

FIG. 4B illustrates the device 400 in a first blood-flow restriction state. The first restriction member 430A is disposed in the distal deployed state defining a radially-expanded diameter 432A. The radially-expanded diameter 432A defines a first annular blood-flow area 435A in combination with the blood-vessel wall 63. Constraining the blood flow 65 to pass through the first annular blood-flow area 435A defines a first blood-flow restriction amount.

FIG. 4C illustrates the device 400 in a second blood-flow restriction state. Although not required, the first restriction member 430A may be disposed in the distal deployed state. The second restriction member 430B is disposed in the distal deployed state defining a radially-expanded diameter 432B. The radially-expanded diameter 432B defines a second annular blood-flow area 435B in combination with the blood-vessel wall 63. Constraining the blood flow 65 to pass through the second annular blood-flow area 435B defines a second blood-flow restriction amount, and the second blood-flow restriction amount may be greater than the first blood-flow restriction amount.

FIG. 4D illustrates the device 400 in a third blood-flow restriction state. Although not required, the first restriction member 430A and the second restriction member 430B may be disposed in the distal deployed state. The third restriction member 430C is disposed in the distal deployed state defining a radially-expanded diameter 432C. The radially-expanded diameter 432C defines a third annular blood-flow area 435C in combination with the blood-vessel wall 63. Constraining the blood flow 65 to pass through the third annular blood-flow area 435C defines a third blood-flow restriction amount, where the third blood-flow restriction amount is greater than the first and second blood-flow restriction amounts.

In the illustrated embodiment, each the restriction members 430A-430C are shown having a cone shape. In other embodiments, any or all of the restriction members 430A-430C may be formed of a different shape, such as a bullet shape, a basket shape, or any other suitable shape for defining the radially-expanded diameters 432A-432C, respectively.

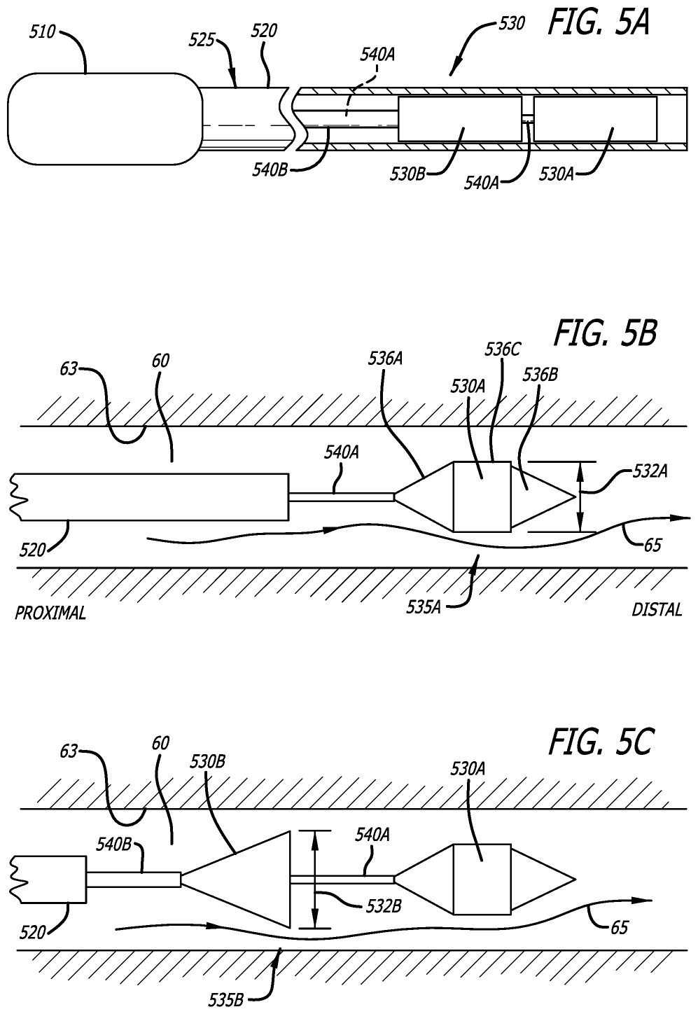

FIGS. 5A-5C illustrate another embodiment of a blood-flow restriction device. FIG. 5A illustrates a cross-sectional side view of the blood flow regulation device (device) 500 in a ready-to-use state. The device 500 generally includes the actuator 510 coupled with the elongate member 520. A blood-flow restriction mechanism 530 of the device 500 includes first restriction member 530A and a second restriction member 530B, where each is shown in the proximal collapsed state within the sheath 525. Each of the first restriction member 530A and the second restriction member 530B is operatively coupled with actuator 510 via a first elongate connection member 540A and a second elongate connection member 540B, respectively. Although not required, the first elongate connection member 540A may be disposed within a lumen of the second elongate connection member 540B.

FIG. 5B illustrates the device 500 in a first blood-flow restriction state. The first restriction member 530A is disposed in the distal deployed state defining a radially-expanded diameter 532A. The radially-expanded diameter 532A defines a first annular blood-flow area 535A in combination with the blood-vessel wall 63. Constraining the blood flow 65 to pass through the first annular blood-flow area 535A defines a first blood-flow restriction amount.

In the illustrated embodiment, the first restriction member 530A has a bullet shape. More specifically, the first restriction member 530A includes a proximal cone portion 536A having a proximally oriented apex and a distal cone portion 536B having a distally oriented apex. The distal cone portion 536B may be coupled with the proximal cone portion 536A via a cylindrical portion 536C. As may be appreciated by one of ordinary skill, in other embodiments, the first restriction member 530A may be formed a different shape, such as any suitable shape for defining the radially-expanded diameter 532A.

FIG. 5C illustrates the device 500 in a second blood-flow restriction state. Although not required, the first restriction member 530A may be disposed in the distal deployed state. The second restriction member 530B is also disposed in the distal deployed state defining a radially-expanded diameter 532B. The radially-expanded diameter 532B defines a second annular blood-flow area 535B in combination with the blood-vessel wall 63. Constraining the blood flow 65 to pass through the second annular blood-flow area 535B defines a second blood-flow restriction amount, where the second blood-flow restriction amount is different than the first blood-flow restriction amount. In some embodiments, the second blood-flow restriction amount may be greater than the first blood-flow restriction amount

In the illustrated embodiment, the second restriction member 530B is shown having a cone shape with a proximally oriented apex. In other embodiments, the second restriction members 530B may be formed a different shape, such as bullet shape, a basket shape, or any suitable shape for defining the radially-expanded diameter 532B.

It is noted that, for each of the embodiments shown and described above, the direction of the blood flow 65 is illustrated as flowing in the distal direction with respect to the device consistent with an instance where the device is inserted within the blood vessel 60 in the direction of the blood flow 65. As discussed above, in an alternative instance, the device may be inserted within the blood vessel 60 in a direction opposite the blood flow 65. In such as alternative instance, the blood flow 65 flows in a proximal direction with respect to the device.

Methods for restricting/regulating blood flow through a blood vessel may include all or any subset of the following steps or processes.

According to some embodiments, a method may include a step of inserting a catheter into the blood vessel so that the blood-flow regulation mechanism disposed at a distal end of the catheter is disposed at a location within the blood vessel where a restriction of blood flow in desired.

A following step may include deploying the first regulation member of the regulation mechanism away from a collapsed state toward a radially expanded state. The deployment of the first regulation member may define a first blood-flow restriction amount. The method may also include deploying the second regulation member away from its collapsed state toward its radially expanded state. The deployment of the second regulation member may define a second blood-flow restriction amount less than the first blood-flow restriction amount.

According to some embodiments, the device may be inserted in the vasculature so that the blood-flow restriction mechanism is disposed within the superior vena cava of the patient, and so that the blood-flow through the superior vena cava may be restricted up to or more than 90 percent.

According to other embodiments, the device may be inserted in the vasculature so that the blood-flow restriction mechanism is disposed within the inferior vena cava of the patient, and so that the blood-flow through the inferior vena cava may be restricted up to or about 50 percent.

A step of the method may also include deploying a third regulation member of the regulation mechanism away from a collapsed state toward a radially expanded state. The deployment of the third regulation member may define a third blood-flow restriction amount less than the second blood-flow restriction amount.

According to some embodiments, an outside diameter of each of the first, second, and third regulation members defines a respective annular blood-flow passageway in combination a wall of the blood vessel, and during use blood flow passes by the respective regulation member via the respective annular blood-flow passageway.

According to further method embodiments, a step may include inserting a catheter into the blood vessel so that the blood-flow regulation mechanism is positioned at a location within the blood vessel where a restriction of blood flow in desired.

A following step may include deploying a first regulation member of the regulation mechanism away from a collapsed state toward a radially expanded state. In such embodiments, the first regulation member may be cone shaped having first conical wall extending between a proximal apex portion and an open distal end.

Another step may include deploying a second regulation member of the regulation mechanism away from a collapsed state toward a radially expanded state. The second regulation member may also be cone shaped having a second conical wall extending between a proximal apex portion and an open distal end.

The first conical wall includes a number of apertures and the first conical wall extends radially outward to a wall of the blood vessel (i.e., fills the blood vessel) so that blood flow through the blood vessel is constrained to passes through the number of apertures.

Further, the first regulation member is positionably disposed within the second regulation member so that the second conical wall may occlude at least a portion of the number of apertures to restrict the blood flow through the blood vessel.

The method may further include a step of adjusting the position of the first regulation member with respect to the second regulation member to adjust the amount of occlusion of the number of apertures by the second conical wall. The step may include adjusting the position of the first regulation member with respect to the second regulation member between a first position and a second position to adjust amount of occlusion, thereby adjusting the restriction of the blood flow between a first restriction amount and a second restriction amount.

In some embodiments of the method, the actuator may receive a signal from the sensor and the logic of the actuator may adjust the position of the first regulation member with respect to the second regulation member based on a signal from the sensor.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. A catheter, comprising:
an elongate member configured for advancement along a blood vessel, the elongate member extending between a proximal end and a distal end;
an actuator disposed at the proximal end, and
a restriction member disposed at the distal end, the restriction member operatively coupled with the actuator via an elongate connection member extending along the elongate member, wherein:
the restriction member:
is configured to transition between a collapsed state and a deployed state in response to an action by the actuator, and
defines a cone in the deployed state, the cone having a conical wall extending radially outward and distally away from an apex portion of the cone,
the conical wall includes a number of apertures extending through the conical wall,
the conical wall is configured to extend radially outward to a blood vessel wall so that blood flow within the blood vessel is constrained to pass through the number of apertures, and
the number of apertures are sized to define a restriction of the blood flow.

2. The catheter of claim 1, wherein at least a portion of the restriction member includes an anti-thrombotic coating.

3. The catheter of claim 1, wherein:
in the collapsed state, the restriction member is disposed within a sheath of the catheter, and
in the deployed state, the restriction member is distally extended away from the distal end of the elongate member.

4. The catheter of claim 1, wherein:
the catheter is configured for placement within a superior vena cava of a patient, and
when deployed within the superior vena cava, the restriction member restricts at least 90 percent of the blood flow through the superior vena cava.

5. The catheter of claim 1, wherein:
the catheter is configured for placement within an inferior vena cava of the patient, and
when deployed within the inferior vena cava, the restriction member restricts approximately 50 percent of the blood flow through the inferior vena cava.

6. The catheter of claim 1, wherein the action is performed via manual manipulation of the actuator by a clinician.

7. A blood flow regulation device, comprising:
an elongate member configured for advancement along a blood vessel, the elongate member extending between a proximal end and a distal end;
a blood-flow regulation mechanism disposed at the distal end of the elongate member; and
an actuator disposed at the proximal end of the elongate member, the actuator operatively coupled with the regulation mechanism,
wherein:
the regulation mechanism is configured to transition between a non-restriction state and a blood-flow restriction state in response to an action by the actuator,
the regulation mechanism includes:
a first regulation member operatively coupled with the actuator via a first elongate connection member extending along the elongate member, and
a second regulation member operatively coupled with the actuator via a second elongate connection member extending along the elongate member,
in the non-restriction state, each of the first and second regulation members are disposed in a collapsed state,
in the blood-flow restriction state, each of the first and second regulation members are disposed in an expanded state, and
the first regulation member is positioned proximal the second regulation member.

8. The device of claim 7, wherein at least a portion of the device includes an anti-thrombotic coating.

9. The device of claim 7, wherein:
the device is configured for placement within a superior vena cava of a patient, and
the blood-flow regulation mechanism is configured to restrict at least 90 percent of the blood flow through the superior vena cava.

10. The device of claim 7, wherein:
the device is configured for placement within an inferior vena cava of the patient, and
blood-flow regulation mechanism is configured to restrict approximately 50 percent of the blood flow through the inferior vena cava.

11. The device of claim 7, wherein:
in the collapsed state each of the first and second regulation members is disposed within a sheath of the elongate member, and
in the expanded state each of the first and second regulation members is distally extended away from the distal end of the elongate member.

12. The device of claim 7, wherein:
the blood-flow regulation mechanism is transitionable between a first blood-flow restriction state configured to restrict the blood flow a first restriction amount and a second blood-flow restriction state configured to restrict the blood flow a second restriction amount, and
the second restriction amount is different from the first restriction amount.

13. The device of claim 12, wherein:
the first blood-flow restriction state is defined by a first relative position between the first and second regulation members in the expanded state,
the second blood-flow restriction state is defined by a second relative position between the first and second regulation members in the expanded state, and
the second relative position is different from the first relative position.

14. The device of claim 12, wherein each of the first and second regulation members defines a hollow cone having a proximal apex portion and a distal open end in the expanded state.

15. The device of claim 14, wherein:
the second regulation member includes a number of second apertures extending through a second conical wall of the second regulation member, and
the second conical wall is configured to extend radially outward to a wall of the blood vessel so that the blood flow is constrained to pass through the number of second apertures.

16. The device of claim 15, wherein in the second relative position, the second regulation member is at least partially disposed within the first regulation member so that at least portion of the number of second apertures is occluded by a first conical wall of the first regulation member.

17. The device of claim 15, wherein:
the first regulation member includes a number of first apertures extending through the first conical wall,
the second regulation member is disposed within the first regulation member so that the second conical wall overlaps the first conical wall,
in the first relative position, the second regulation member is rotatably positioned relative to the first regulation member so that the number of first apertures overlaps the number of second apertures a first overlapping amount,
in the second relative position, the second regulation member is rotatably positioned relative to the first regulation member so that the number of first apertures overlaps the number of second apertures a second overlapping amount, and
the second overlapping amount is different from the first overlapping amount.

18. The device of claim 13, wherein:
in the first blood-flow restriction state, the second regulation member is in the expanded state such that a second conical wall of the second regulation member is radially expanded to define an annular second passageway between the second regulation member and the wall of the blood vessel, and
the annular second passageway is sized to define the first blood-flow restriction amount.

19. The device of claim 18, wherein:
in the second blood-flow restriction state, the first regulation member is in the expanded state such that a first conical wall of the first regulation member is radially expanded to define an annular first passageway between the first regulation member and the wall of the blood vessel, and
the annular first passageway is sized to define the second blood-flow restriction amount.

20. The device of claim 19, wherein the second blood-flow restriction amount is greater than the first blood-flow restriction amount

21. The device of claim 18, wherein:
the second regulation member defines a bullet shape in the distal deployed state, and
the bullet shape includes a cone shaped proximal portion having a proximally oriented apex and a cone shaped distal portion having a distally oriented apex.

22. The device of claim 18, wherein:
the blood-flow regulation mechanism is further transitionable to a third blood-flow restriction state configured to restrict the blood flow a third restriction amount, and
the third restriction amount is less than the first restriction amount and the second restriction amount.

23. The device of claim 22, wherein:
the regulation mechanism further includes a third regulation member operatively coupled with the actuator via a third elongate connection member extending along the elongate member,
the third regulation member is positioned distal the first and second regulation members,
in the non-restriction state, the third regulation member is disposed in a collapsed state within the sheath,
in the third blood-flow restriction state, the third restriction member is disposed in an expanded state such that a third conical wall of the first regulation member is radially expanded to define an annular third passageway between the third regulation member and the wall of the blood vessel, and
the annular third passageway is sized to define the third blood-flow restriction amount.

24. The device of claim 12, wherein:
the actuator includes a controller including controller logic, and
an electro-mechanical actuating mechanism coupled between the controller and the first and second elongate connection members,
the actuator is configured to variably transition the regulation mechanism between the first blood-flow restriction state and the second blood-flow restriction state.

25. The device of claim 24, further comprising:
a sensor positioned along the elongate member and coupled with the controller, the sensor configured to provide a signal to the controller based on one or more of a static pressure, a dynamic pressure, or a velocity of the blood within the blood vessel,
wherein the logic is configured to variably transition the regulation mechanism in response to the signal from the sensor.

26-33. (canceled)
